SEQUENCE LISTING (8 sequences)

SEQ ID NO: 1 - DNA, 6389 bp, synthetic construct
  source 1..6389: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatgagcatccaggtggagcatcccgcaggaggctataaaaagctgtttgagactgtcgaagaactgagtagccctctgaccgcacacgtgaccggccgcatcccactgtggctgacaggctctctgctgagatgcggccccggcctgttcgaagtgggcagcgagcctttctaccacctgtttgacggccaggccctgctgcacaagttcgacttcaaggagggccacgtgacctaccacaggaggttcatcaggacagacgcctatgtgagagccatgaccgagaagcggatcgtgatcaccgagttcggcacatgcgcctttccagatccctgtaagaatatcttcagccgcttcttttcctactttaggggcgtggaggtgacagacaacgccctggtgaacgtgtaccctgtgggcgaggattactatgcctgcaccgagacaaacttcatcaccaagatcaatccagagactctggagacgatcaagcaggtggacctgtgcaactacgtgagcgtgaatggcgccacagcccacccccacatcgagaacgatggcaccgtgtacaacatcggcaattgcttcggcaagaacttttccatcgcctataatatcgtgaagatcccacctctccaggcagacaaggaggaccccatcagcaagagcgagatcgtggtgcagttcccttgtagcgaccggtttaagccatcttacgtgcacagcttcggcctgacaccaaactatatcgtgtttgtggagacacccgtgaagatcaacctgttcaagttcctgagcagctggagcctgtggggcgccaactacatggactgcttcgagtccaatgagacaatgggcgtgtggctgcacatcgccgataagaagagaaagaagtacctgaacaataagtatcggacatcccccttcaacctgtttcaccacatcaacacctatgaggacaatggctttctgatcgtggatctgtgctgttggaagggcttcgagttcgtgtacaactatctgtacctggccaacctgagagagaattgggaggaggtgaagaagaatgcaaggaaggcacctcagccagaggtgcggcgctacgtgctgccactgaacatcgacaaggccgataccggcaagaacctggtgacactgcccaataccacagccacagccatcctgtgcagcgacgagacaatctggctggagcccgaggtgctgttctctggccctcgccaggccttcgagtttccacagatcaattaccagaagtattgcggcaagccctatacctacgcctatggcctgggcctgaaccacttcgtgcctgacaggctgtgcaagctgaacgtgaaaaccaaggagacatgggtgtggcaggagcctgactcctacccctctgagcctatcttcgtgagccacccagatgccctggaggaggacgatggcgtggtgctgtccgtggtggtgtctccaggcgcaggacagaagccagcatatctgctgatcctgaacgccaaggacctgtccgaggtggccagagcagaggtcgaaatcaatatccctgtcacattccacgggctgttcaagaaatcctgaaagcttgcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 2 - DNA, 6391 bp, synthetic construct
  source 1..6391: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccgccaccatgtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaatgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 3 - DNA, 20 bp, synthetic construct
  source 1..20: mol_type = other DNA
agcgagcctttctaccacct

SEQ ID NO: 4 - DNA, 20 bp, synthetic construct
  source 1..20: mol_type = other DNA
atggctctcacataggcgtc

SEQ ID NO: 5 - DNA, 20 bp, synthetic construct
  source 1..20: mol_type = other DNA
catcctgctggtggttacaa

SEQ ID NO: 6 - DNA, 20 bp, synthetic construct
  source 1..20: mol_type = other DNA
acaggtggtaaaatggctca

SEQ ID NO: 7 - DNA, 21 bp, synthetic construct
  source 1..21: mol_type = other DNA
agtcccttgccatcctaaaag

SEQ ID NO: 8 - DNA, 20 bp, synthetic construct
  source 1..20: mol_type = other DNA
caatgctatcacctcccctg

(19) United States
(12) Patent Application Publication
(10) Pub. No.: US 2025/0295808 A1
(43) Pub. Date: Sep. 25, 2025

(54) A SYSTEM FOR AN OCULAR GENE THERAPY AND A PROCESS FOR PREPARING THEREOF

(71) Applicant: INDIAN INSTITUTE OF TECHNOLOGY KANPUR, KANPUR (IN)

(72) Inventors: Jayandharan Giridhara Rao, Kanpur (IN); Vijayata Singh, Kanpur (IN); Shamshul Huda, Kanpur (IN); G. Narendra Kumar, Kanpur (IN)

(21) Appl. No.: 18/848,602

(22) PCT Filed: Mar. 28, 2023
(86) PCT No.: PCT/IN2023/050295

§ 371 Date: Sep. 19, 2024

(30) Foreign Application Priority Data
Mar. 28, 2022 (IN) 202211017896

(51) Int. Cl. A61K 48/00; A61K 38/46; A61P 27/02; C12N 9/16; C12N 15/86

(52) U.S. Cl. CPC A61K 48/005; A61K 38/465; A61K 48/0091; A61P 27/02; C12N 9/16; C12N 15/86; C12Y 301/01064; C12N 2750/14143; C12N 2750/14152; C12N 2800/22; C12Y 301/30002

(57) ABSTRACT

A system for an ocular gene therapy is provided. The system includes one or more optimized transgenes. The one or more optimized transgenes include pAAV.CMV.CodOpt.RPE65 and pAAV.CMV.Kozak.RPE65. The optimized transgenes pAAV.CMV.CodOpt.RPE65 and pAAV.CMV.Kozak.RPE65 have shown to exhibit enhanced RPE65 gene expression when compared to wild type RPE65 gene transfer in suitable models. These optimized genes may enhance therapeutic response during LCA2 gene therapy. The present invention also provides a process for preparing the optimized transgene for an ocular gene therapy.

### EARLIEST PRIORITY DATE

This application claims priority from a Provisional patent application filed in India having patent application No. 202211017896, filed on Mar. 28, 2022, and a PCT application bearing application no. PCT/IN2023/050295, filed on Mar. 28, 2023.

### FIELD OF INVENTION

Embodiment of the present invention relates to ocular gene therapy techniques and more particularly, it relates to a system for an ocular gene therapy and a process for preparing thereof.

### BACKGROUND

Leber congenital amaurosis 2 (LCA2) is an inherited retinal degenerative disorder caused due to mutations in the RPE65 gene. The LCA2 results in severe visual impairment during childhood, which deteriorates over time leading to complete blindness. Unfortunately, there is currently no cure for LCA 2. However, the development of gene replacement therapies and other potential new treatments are offering hope for patients.

Recently, research on gene therapy for the LCA2 using adeno-associated virus (AAV) vectors have shown therapeutic response in the patients with LCA2 by delivering functional retinal pigment epithelium-65 kda protein-encoding gene (RPE65). Clinical trials have shown efficacy of RPE65 replacement therapy using AAV2 vectors, mainly for the first three years but not long-term.

Thus, strategies to increase gene transfer potential of the AAV2 vectors in the ocular niche for the long-term and increased expression of RPE65 transgene are required. Maurya S et al., 2019 and Büning H et al., 2019, in their papers, disclosed that one such strategy includes capsid engineering by introducing modifications at SUMOylation, neddylation, phosphorylation and ubiquitination sites of the viral capsid, which has shown to enhance the efficacy of the vector.

Hanson G et al., 2018, Frumkin I et al., 2018, and Zarghampoor F et al., 2019, in their papers, explained that augmenting the transgene for improved protein expression has a better therapeutic outcome. Organisms prefer some codons over others resulting in differing frequencies of synonymous codons, a phenomenon known as codon usage bias. Implementing a strategy like codon optimization of a target transgene enhances protein expression.

Mohan R A et al., 2014, in their paper, disclosed that in most eukaryotic cells, Kozak sequence serves as a translation initiation site and mutations in Kozak sequence are reported to change translation status leading to a diseased condition. Therefore, strategies like the codon optimization of the transgene or the addition of Kozak sequence may result in high protein formation.

Hence, there is a need for a system including optimized transgene with Kozak sequence along with RPE65 or codon optimized form of RPE65 for an ocular gene therapy and a process for preparing thereof.

### SUMMARY

In accordance with an embodiment of the present invention, a system for an ocular gene therapy is provided. The system includes one or more optimized transgenes configured to improve efficiency of the ocular gene therapy in Leber congenital amaurosis 2 (LCA2) condition. The one or more optimized transgenes are selected from a group consisting of a nucleotide sequence as set forth in SEQ ID No. 1 with pAAV.CMV.CodOpt.RPE65 and a nucleotide sequence as set forth in SEQ ID No. 2 with pAAV.CMV.Kozak.RPE65.

In accordance with another embodiment of the present invention, a process for preparing an optimized transgene is provided. The process includes transfecting adeno-associated virus (AAV) packaging cell line with AAV-rep/cap, adenoviral helper plasmid (pHelper), and one of codon optimized RPE65 and Kozak sequence containing RPE65. The process also includes harvesting the transfected cell line after a duration of 72 hours. The process includes carrying out cell lysis of the harvested cell line to obtain a cell lysate. The process also includes treating the cell lysate with 25 units/ml of Benzonase. The process further includes purifying the treated cell lysate to obtain the optimized transgene. The optimized transgene comprises one of a nucleotide sequence as set forth in SEQ ID No. 1 with pAAV.CMV.CodOpt.RPE65 and a nucleotide sequence as set forth in SEQ ID No. 2 with pAAV.CMV.Kozak.RPE65.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a schematic representation of pAAV.CMV.CodOpt.RPE65, in accordance with an embodiment of the present invention;

FIG. 2 is a schematic representation of pAAV.CMV.Kozak.RPE65, in accordance with an embodiment of the present invention;

FIG. 3 represents a flowchart including steps of a process for preparing an optimized transgene, in accordance with an embodiment of the present invention;

FIG. 4 is a graphical representation of relative RPE65 gene expression for pAAVKozakRPE65 and pAAVCodOptRPE65, in accordance with an embodiment of the present invention;

FIG. 5 represents immunostaining of RPE65 a-b) RPE65 expression post-transduction and c) quantification of integrated density per unit area, post-transduction, in accordance with an embodiment of the present invention;

FIG. 6 represents ERG for test groups after 10 weeks follow-up a) representative ERG waterfall from each group and b) graphical representation of ‘A-wave’ and ‘B-wave’ amplitude, in accordance with an embodiment of the present invention; and

FIG. 7 represents immunostaining of mice eye sections a) RPE65 and b) GFAP staining, in accordance with an embodiment of the present invention.

Further, those skilled in the art will appreciate that elements in the figures are illustrated for simplicity and may not have necessarily been drawn to scale. Furthermore, in terms of the method steps, chemical compounds, and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

The Sequence Listing submitted with the present patent application complies with the requirements of 37 CFR §§ 1.821-1.825. The sequence listing is submitted in [ST.25 text format/ST.26 XML format] and is incorporated herein by reference in its entirety. If previously submitted as part of the international application, the sequence listing remains unchanged and fully compliant with U.S. national requirements.

Embodiment of the present invention a system for an ocular gene therapy. The invention mainly focuses on development of codon optimized RPE65 and Kozak sequence containing RPE65 vectors for gene delivery in Leber congenital amaurosis 2 (LCA2).

As used herein the term “ocular gene therapy” refers to a promising and emerging field with the potential to treat both rare IRDs and more common acquired retinal conditions. Newer generations of viral and synthetic vectors may improve expressivity and carrying capacity while reducing immunogenicity and mutagenicity.

In an embodiment, the system for an ocular gene therapy is provided. The system includes one or more optimized transgenes configured to improve efficiency of the ocular gene therapy in Leber congenital amaurosis 2 (LCA2) condition. The one or more optimized transgenes are selected from a group consisting of a nucleotide sequence as set forth in SEQ ID No. 1 with pAAV.CMV.CodOpt.RPE65 and a nucleotide sequence as set forth in SEQ ID No. 2 with pAAV.CMV.Kozak.RPE65.

As used herein the term “transgene” refers to a gene that has been transferred by a number of genetic engineering techniques, from one organism to another.

The optimized transgene including pAAV.CMV.CodOpt.RPE65 vector sequence is as follows:

SEQ ID No. 1
AGCGCCCAATACGCAAACCGCCTCTCCCCGCGCGTTGGCCGATTCATTAATGC
AGCTGGCACGACAGGTTTCCCGACTGGAAAGCGGGCAGTGAGCGCAACGCA
ATTAATGTGAGTTAGCTCACTCATTAGGCACCCCAGGCTTTACACTTTATGCT
TCCGGCTCGTATGTTGTGTGGAATTGTGAGCGGATAACAATTTCACACAGGA
AACAGCTATGACCATGATTACGCCAGATTTAATTAAGGCTGCGCGCTCGCTCG
CTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCA
CTAGGGGTTCCTTGTAGTTAATGATTAACCCGCCATGCTACTTATCTACGTAG
CCATGCTCTAGGAAGATCGGAATTCGCCCTTAAGCTAGCTAGTTATTAATAGT
AATCAATTACGGGGTCATTAGTTCATAGCCCATATATGGAGTTCCGCGTTAC
ATAACTTACGGTAAATGGCCCGCCTGGCTGACCGCCCAACGACCCCCGC
CCATTGACGTCAATAATGACGTATGTTCCCATAGTAACGCCAATAGGGAC
TTTCCATTGACGTCAATGGGTGGAGTATTTACGGTAAACTGCCCACTTGG
CAGTACATCAAGTGTATCATATGCCAAGTACGCCCCCTATTGACGTCAAT
GACGGTAAATGGCCCGCCTGGCATTATGCCCAGTACATGACCTTATGGG
ACTTTCCTACTTGGCAGTACATCTACGTATTAGTCATCGCTATTACCATG
GTGATGCGGTTTTGGCAGTACATCAATGGGCGTGGATAGCGGTTTGACT
CACGGGGATTTCCAAGTCTCCACCCCATTGACGTCAATGGGAGTTTGTTT
TGGCACCAAAATCAACGGGACTTTCCAAAATGTCGTAACAACTCCGCCCC
ATTGACGCAAATGGGCGGTAGGCGTGTACGGTGGGAGGTCTATATAAGC
AGAGCTGGTTTAGTGAACCGTCAGATCCTGCAGAAGTTGGTCGTGAGGCACT
GGGCAGGTAAGTATCAAGGTTACAAGACAGGTTTAAGGAGACCAATAGAAA
CTGGGCTTGTCGAGACAGAGAAGACTCTTGCGTTTCTGATAGGCACCTATTGG
TCTTACTGACATCCACTTTGCCTTTCTCTCCACAGGTGTCCAGGCGGCCGCCA
TGAGCATCCAGGTGGAGCATCCCGCAGGAGGCTATAAAAAGCTGTTTGAGAC
TGTCGAAGAACTGAGTAGCCCTCTGACCGCACACGTGACCGGCCGCATCCCA
CTGTGGCTGACAGGCTCTCTGCTGAGATGCGGCCCCGGCCTGTTCGAAGTGG
GCAGCGAGCCTTTCTACCACCTGTTTGACGGCCAGGCCCTGCTGCACAAGTTC
GACTTCAAGGAGGGCCACGTGACCTACCACAGGAGGTTCATCAGGACAGACG
CCTATGTGAGAGCCATGACCGAGAAGCGGATCGTGATCACCGAGTTCGGCAC
ATGCGCCTTTCCAGATCCCTGTAAGAATATCTTCAGCCGCTTCTTTTCCTACTT
TAGGGGCGTGGAGGTGACAGACAACGCCCTGGTGAACGTGTACCCTGTGGGC
GAGGATTACTATGCCTGCACCGAGACAAACTTCATCACCAAGATCAATCCAG
AGACTCTGGAGACGATCAAGCAGGTGGACCTGTGCAACTACGTGAGCGTGAA
TGGCGCCACAGCCCACCCCCACATCGAGAACGATGGCACCGTGTACAACATC
GGCAATTGCTTCGGCAAGAACTTTTCCATCGCCTATAATATCGTGAAGATCCC
ACCTCTCCAGGCAGACAAGGAGGACCCCATCAGCAAGAGCGAGATCGTGGTG
CAGTTCCCTTGTAGCGACCGGTTTAAGCCATCTTACGTGCACAGCTTCGGCCT
GACACCAAACTATATCGTGTTTGTGGAGACACCCGTGAAGATCAACCTGTTC
AAGTTCCTGAGCAGCTGGAGCCTGTGGGGCGCCAACTACATGGACTGCTTCG
AGTCCAATGAGACAATGGGCGTGTGGCTGCACATCGCCGATAAGAAGAGAA
AGAAGTACCTGAACAATAAGTATCGGACATCCCCCTTCAACCTGTTTCACCAC
ATCAACACCTATGAGGACAATGGCTTTCTGATCGTGGATCTGTGCTGTTGGAA
GGGCTTCGAGTTCGTGTACAACTATCTGTACCTGGCCAACCTGAGAGAGAATT
GGGAGGAGGTGAAGAAGAATGCAAGGAAGGCACCTCAGCCAGAGGTGCGGC
GCTACGTGCTGCCACTGAACATCGACAAGGCCGATACCGGCAAGAACCTGGT
GACACTGCCCAATACCACAGCCACAGCCATCCTGTGCAGCGACGAGACAATC
TGGCTGGAGCCCGAGGTGCTGTTCTCTGGCCCTCGCCAGGCCTTCGAGTTTCC
ACAGATCAATTACCAGAAGTATTGCGGCAAGCCCTATACCTACGCCTATGGC
CTGGGCCTGAACCACTTCGTGCCTGACAGGCTGTGCAAGCTGAACGTGAAAA
CCAAGGAGACATGGGTGTGGCAGGAGCCTGACTCCTACCCCTCTGAGCCTAT
CTTCGTGAGCCACCCAGATGCCCTGGAGGAGGACGATGGCGTGGTGCTGTCC
GTGGTGGTGTCTCCAGGCGCAGGACAGAAGCCAGCATATCTGCTGATCCTGA
ACGCCAAGGACCTGTCCGAGGTGGCCAGAGCAGAGGTCGAAATCAATATCCC
TGTCACATTCCACGGGCTGTTCAAGAAATCCTGAAAGCTTGCTTGGATCCAAT
CAACCTCTGGATTACAAAATTTGTGAAAGATTGACTGGTATTCTTAACTATGT
TGCTCCTTTTACGCTATGTGGATACGCTGCTTTAATGCCTTTGTATCATGCTAT
TGCTTCCCGTATGGCTTTCATTTTCTCCTCCTTGTATAAATCCTGGTTGCTGTC
TCTTTATGAGGAGTTGTGGCCCGTTGTCAGGCAACGTGGCGTGGTGTGCACTG
TGTTTGCTGACGCAACCCCCACTGGTTGGGGCATTGCCACCACCTGTCAGCTC
CTTTCCGGGACTTTCGCTTTCCCCCTCCCTATTGCCACGGCGGAACTCATCGCC
GCCTGCCTTGCCCGCTGCTGGACAGGGGCTCGGCTGTTGGGCACTGACAATTC
CGTGGTGTTGTCGGGGAAGCTGACGTCCTTTCCATGGCTGCTCGCCTGTGTTG
CCACCTGGATTCTGCGCGGGACGTCCTTCTGCTACGTCCCTTCGGCCCTCAAT
CCAGCGGACCTTCCTTCCCGCGGCCTGCTGCCGGCTCTGCGGCCTCTTCCGCG
TCTTCGAGATCTGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCT
GGGGGGTGGGGTGGGGCAGGACAGCAAGGGGGAGGATTGGGAAGACAATAG
CAGGCATGCTGGGGACTCGAGTTAAGGGCGAATTCCCGATTAGGATCTTCCT
AGAGCATGGCTACGTAGATAAGTAGCATGGCGGGTTAATCATTAACTACAAG
GAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCC
TCAGTGAGCGAGCGAGCGCGCAGCCTTAATTAACCTAATTCACTGGCCGTCG
TTTTACAACGTCGTGACTGGGAAAACCCTGGCGTTACCCAACTTAATCGCCTT
GCAGCACATCCCCCTTTCGCCAGCTGGCGTAATAGCGAAGAGGCCCGCACCG
ATCGCCCTTCCCAACAGTTGCGCAGCCTGAATGGCGAATGGGACGCGCCCTG
TAGCGGCGCATTAAGCGCGGCGGGTGTGGTGGTTACGCGCAGCGTGACCGCT
ACACTTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTTAGG
GTTCCGATTTAGTGCTTTACGGCACCTCGACCCCAAAAAACTTGATTAGGGTG
ATGGTTCACGTAGTGGGCCATCGCCCCGATAGACGGTTTTTCGCCCTTTGACG
CTGGAGTTCACGTTCCTCAATAGTGGACTCTTGTTCCAAACTGGAACAACACT
CAACCCTATCTCGGTCTATTCTTTTGATTTATAAGGGATTTTTCCGATTTCGGC
CTATTGGTTAAAAAATGAGCTGATTTAACAAAAATTTAACGCGAATTTTAACA
AAATATTAACGTTTATAATTTCAGGTGGCATCTTTCGGGGAAATGTGCGCGGA
ACCCCTATTTGTTTATTTTTCTAAATACATTCAAATATGTATCCGCTCATGAGA
CAATAACCCTGATAAATGCTTCAATAATATTGAAAAAGGAAGAGTATGAGTA
TTCAACATTTCCGTGTCGCCCTTATTCCCTTTTTTGCGGCATTTTGCCTTCCTGT
TTTTGCTCACCCAGAAACGCTGGTGAAAGTAAAAGATGCTGAAGATCAGTTG
GGTGCACGAGTGGGTTACATCGAACTGGATCTCAATAGTGGTAAGATCCTTG
AGAGTTTTCGCCCCGAAGAACGTTTTCCAATGATGAGCACTTTTAAAGTTCTG
CTATGTGGCGCGGTATTATCCCGTATTGACGCCGGGCAAGAGCAACTCGGTC
GCCGCATACACTATTCTCAGAATGACTTGGTTGAGTACTCACCAGTCACAGAA
AAGCATCTTACGGATGGCATGACAGTAAGAGAATTATGCAGTGCTGCCATAA
CCATGAGTGATAACACTGCGGCCAACTTACTTCTGACAACGATCGGAGGACC
GAAGGAGCTAACCGCTTTTTTGCACAACATGGGGGATCATGTAACTCGCCTTG
ATCGTTGGGAACCGGAGCTGAATGAAGCCATACCAAACGACGAGCGTGACAC
CACGATGCCTGTAGTAATGGTAACAACGTTGCGCAAACTATTAACTGGCGAA
CTACTTACTCTAGCTTCCCGGCAACAATTAATAGACTGGATGGAGGCGGATA
AAGTTGCAGGACCACTTCTGCGCTCGGCCCTTCCGGCTGGCTGGTTTATTGCT
GATAAATCTGGAGCCGGTGAGCGTGGGTCTCGCGGTATCATTGCAGCACTGG
GGCCAGATGGTAAGCCCTCCCGTATCGTAGTTATCTACACGACGGGGAGTCA
GGCAACTATGGATGAACGAAATAGACAGATCGCTGAGATAGGTGCCTCACTG
ATTAAGCATTGGTAACTGTCAGACCAAGTTTACTCATATATACTTTAGATTGA
TTTAAAACTTCATTTTTAATTTAAAAGGATCTAGGTGAAGATCCTTTTTGATA
ATCTCATGACCAAAATCCCTTAACGTGAGTTTTCGTTCCACTGAGCGTCAGAC
CCCGTAGAAAAGATCAAAGGATCTTCTTGAGATCCTTTTTTTCTGCGCGTAAT
CTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGC
AGATACCAAATACTGTCCTTCTAGTGTAGCCGTAGTTAGGCCACCACTTCAAG
AACTCTGTAGCACCGCCTACATACCTCGCTCTGCTAATCCTGTTACCAGTGGC
TGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCT
ATGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAA
CGCCTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGCAA
CGCGGCCTTTTTACGGTTCCTGGCCTTTTGCTGCGGTTTTGCTCACATGTTCTT
TCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGA
GGAAGCGGAAG

### Key:

Bold font: CMV Enhancer/Promoter
Underlined font: Codon Optimized RPE65 gene

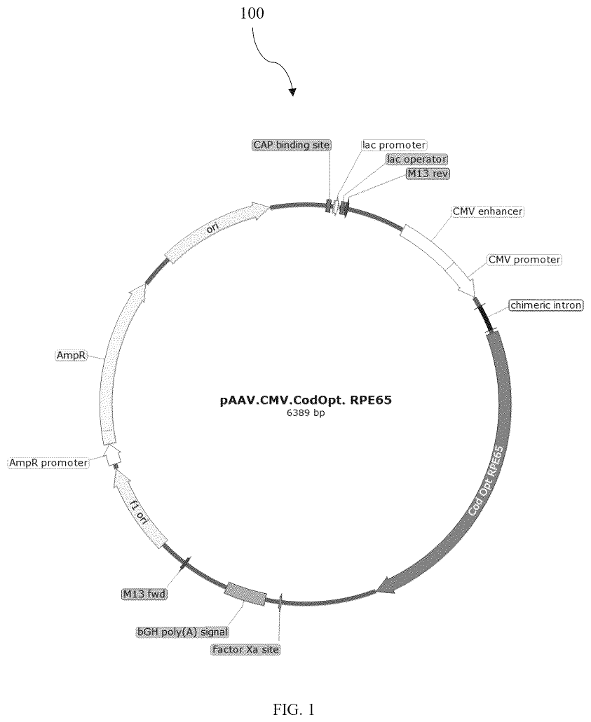

FIG. 1 is a schematic representation of pAAV.CMV.CodOpt.RPE65, in accordance with an embodiment of the present invention.

The optimized transgene including pAAV.CMV.Kozak.RPE65 vector sequence is as follows:

SEQ ID No. 2
AGCGCCCAATACGCAAACCGCCTCTCCCCGCGCGTTGGCCGATTCATTAATGC
AGCTGGCACGACAGGTTTCCCGACTGGAAAGCGGGCAGTGAGCGCAACGCA
ATTAATGTGAGTTAGCTCACTCATTAGGCACCCCAGGCTTTACACTTTATGCT
TCCGGCTCGTATGTTGTGTGGAATTGTGAGCGGATAACAATTTCACACAGGA
AACAGCTATGACCATGATTACGCCAGATTTAATTAAGGCTGCGCGCTCGCTCG
CTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCA
CTAGGGGTTCCTTGTAGTTAATGATTAACCCGCCATGCTACTTATCTACGTAG
CCATGCTCTAGGAAGATCGGAATTCGCCCTTAAGCTAGCTAGTTATTAATAGT
AATCAATTACGGGGTCATTAGTTCATAGCCCATATATGGAGTTCCGCGTTACA
TAACTTACGGTAAATGGCCCGCCTGGCTGACCGCCCAACGACCCCCGCCCATT
GACGTCAATAATGACGTATGTTCCCATAGTAACGCCAATAGGGACTTTCCATT
GACGTCAATGGGTGGAGTATTTACGGTAAACTGCCCACTTGGCAGTACATCA
AGTGTATCATATGCCAAGTACGCCCCCTATTGACGTCAATGACGGTAAATGG
CCCGCCTGGCATTATGCCCAGTACATGACCTTATGGGACTTTCCTACTTGGCA
GTACATCTACGTATTAGTCATCGCTATTACCATGGTGATGCGGTTTTGGCAGT
ACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTC
CAAAATGTCGTAACAACTCCGCCCCATTGACGCAAATGGGCGGTAGGCGTGT
ACGGTGGGAGGTCTATATAAGCAGAGCTGGTTTAGTGAACCGTCAGATCCTG
CAGAAGTTGGTCGTGAGGCACTGGGCAGGTAAGTATCAAGGTTACAAGACAG
GTTTAAGGAGACCAATAGAAACTGGGCTTGTCGAGACAGAGAAGACTCTTGC
GTTTCTGATAGGCACCTATTGGTCTTACTGACATCCACTTTGCCTTTCTCTCCA
CAGGTGTCCAGGCGGCCGCGCCGCCACCATGTCTATCCAGGTTGAGCATCCTG
CTGGTGGTTACAAGAAACTGTTTGAAACTGTGGAGGAACTGTCCTCGCCGCTCACA
GCTCATGTAACAGGCAGGATCCCCCTCTGGCTCACCGGCAGTCTCCTTCGATGTG
GGCCAGGACTCTTTGAAGTTGGATCTGAGCCATTTTACCACCTGTTTGATGGGCAA
GCCCTCCTGCACAAGTTTGACTTTAAAGAAGGACATGTCACATACCACAGAAGGTT
CATCCGCACTGATGCTTACGTACGGGCAATGACTGAGAAAAGGATCGTCATAACAG
AATTTGGCACCTGTGCTTTCCCAGATCCCTGCAAGAATATATTTTCCAGGTTTTTTTC
TTACTTTCGAGGAGTAGAGGTTACTGACAATGCCCTTGTTAATGTCTACCCAGTGG
GGGAAGATTACTACGCTTGCACAGAGACCAACTTTATTACAAAGATTAATCCAGAGA
CCTTGGAGACAATTAAGCAGGTTGATCTTTGCAACTATGTCTCTGTCAATGGGGCC
ACTGCTCACCCCCACATTGAAAATGATGGAACCGTTTACAATATTGGTAATTGCTTT
GGAAAAAATTTTTCAATTGCCTACAACATTGTAAAGATCCCACCACTGCAAGCAGAC
AAGGAAGATCCAATAAGCAAGTCAGAGATCGTTGTACAATTCCCCTGCAGTGACCG
ATTCAAGCCATCTTACGTTCATAGTTTTGGTCTGACTCCCAACTATATCGTTTTTGTG
GAGACACCAGTCAAAATTAACCTGTTCAAGTTCCTTTCTTCATGGAGTCTTTGGGGA
GCCAACTACATGGATTGTTTTGAGTCCAATGAAACCATGGGGGTTTGGCTTCATATT
GCTGACAAAAAAAGGAAAAAGTACCTCAATAATAAATACAGAACTTCTCCTTTCAAC
CTCTTCCATCACATCAACACCTATGAAGACAATGGGTTTCTGATTGTGGATCTCTGC
TGCTGGAAAGGATTTGAGTTTGTTTATAATTACTTATATTTAGCCAATTTACGTGAGA
ACTGGGAAGAGGTGAAAAAAAATGCCAGAAAGGCTCCCCAACCTGAAGTTAGGAG
ATATGTACTTCCTTTGAATATTGACAAGGCTGACACAGGCAAGAATTTAGTCACGCT
CCCCAATACAACTGCCACTGCAATTCTGTGCAGTGACGAGACTATCTGGCTGGAGC
CTGAAGTTCTCTTTTCAGGGCCTCGTCAAGCATTTGAGTTTCCTCAAATCAATTACC
AGAAGTATTGTGGGAAACCTTACACATATGCGTATGGACTTGGCTTGAATCACTTTG
TTCCAGATAGGCTCTGTAAGCTGAATGTCAAAACTAAAGAAACTTGGGTTTGGCAAG
AGCCTGATTCATACCCATCAGAACCCATCTTTGTTTCTCACCCAGATGCCTTGGAAG
AAGATGATGGTGTAGTTCTGAGTGTGGTGGTGAGCCCAGGAGCAGGACAAAAGCC
TGCTTATCTCCTGATTCTGAATGCCAAGGACTTAAGTGAAGTTGCCCGGGCTGAAG
TGGAGATTAACATCCCTGTCACCTTTCATGGACTGTTCAAAAAATCTTGAGCTTGGA
TCCAATCAACCTCTGGATTACAAAATTTGTGAAAGATTGACTGGTATTCTTAA
CTATGTTGCTCCTTTTACGCTATGTGGATACGCTGCTTTAATGCCTTTGTATCA
TGCTATTGCTTCCCGTATGGCTTTCATTTTCTCCTCCTTGTATAAATCCTGGTT
GCTGTCTCTTTATGAGGAGTTGTGGCCCGTTGTCAGGCAACGTGGCGTGGTGT
GCACTGTGTTTGCTGACGCAACCCCCACTGGTTGGGGCATTGCCACCACCTGT
CAGCTCCTTTCCGGGACTTTCGCTTTCCCCCTCCCTATTGCCACGGCGGAACTC
ATCGCCGCCTGCCTTGCCCGCTGCTGGACAGGGGCTCGGCTGTTGGGCACTGA
CAATTCCGTGGTGTTGTCGGGGAAGCTGACGTCCTTTCCATGGCTGCTCGCCT
GTGTTGCCACCTGGATTCTGCGCGGGACGTCCTTCTGCTACGTCCCTTCGGCC
CTCAATCCAGCGGACCTTCCTTCCCGCGGCCTGCTGCCGGCTCTGCGGCCTCT
TCCGCGTCTTCGAGATCTGCCTCGACTGTGCCTTCTAGTTGCCAGCCATCTGTT
GTTTGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTCATT
CTATTCTGGGGGGTGGGGTGGGGCAGGACAGCAAGGGGGAGGATTGGGAAG
ACAATAGCAGGCATGCTGGGGACTCGAGTTAAGGGCGAATTCCCGATTAGGA
TCTTCCTAGAGCATGGCTACGTAGATAAGTAGCATGGCGGGTTAATCATTAAC
TACAAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGG
GCGGCCTCAGTGAGCGAGCGAGCGCGCAGCCTTAATTAACCTAATTCACTGG
CCGTCGTTTTACAACGTCGTGACTGGGAAAACCCTGGCGTTACCCAACTTAAT
CGCCTTGCAGCACATCCCCCTTTCGCCAGCTGGCGTAATAGCGAAGAGGCCC
GCACCGATCGCCCTTCCCAACAGTTGCGCAGCCTGAATGGCGAATGGGACGC
GCCCTGTAGCGGCGCATTAAGCGCGGCGGGTGTGGTGGTTACGCGCAGCGTG
ACCGCTACACTTGCCAGCGCCCTAGCGCCCGCTCCTTTCGCTTTCTTCCCTTCC
TTTCTCGCCACGTTCGCCGGCTTTCCCCGTCAAGCTCTAAATCGGGGGCTCCC
TTTAGGGTTCCGATTTAGTGCTTTACGGCACCTCGACCCCAAAAAACTTGATT
AGGGTGATGGTTCACGTAGTGGGCCATCGCCCCGATAGACGGTTTTTCGCCCT
TTGACGCTGGAGTTCACGTTCCTCAATAGTGGACTCTTGTTCCAAACTGGAAC
AACACTCAACCCTATCTCGGTCTATTCTTTTGATTTATAAGGGATTTTTCCGAT
TTCGGCCTATTGGTTAAAAAATGAGCTGATTTAACAAAAATTTAACGCGAATT
TTAACAAAATATTAACGTTTATAATTTCAGGTGGCATCTTTCGGGGAAATGTG
CGCGGAACCCCTATTTGTTTATTTTTCTAAATACATTCAAATATGTATCCGCTC
ATGAGACAATAACCCTGATAAATGCTTCAATAATATTGAAAAAGGAAGAGTA
TGAGTATTCAACATTTCCGTGTCGCCCTTATTCCCTTTTTTGCGGCATTTTGCC
TTCCTGTTTTTGCTCACCCAGAAACGCTGGTGAAAGTAAAAGATGCTGAAGAT
CAGTTGGGTGCACGAGTGGGTTACATCGAACTGGATCTCAATAGTGGTAAGA
TCCTTGAGAGTTTTCGCCCCGAAGAACGTTTTCCAATGATGAGCACTTTTAAA
GTTCTGCTATGTGGCGCGGTATTATCCCGTATTGACGCCGGGCAAGAGCAACT
CGGTCGCCGCATACACTATTCTCAGAATGACTTGGTTGAGTACTCACCAGTCA
CAGAAAAGCATCTTACGGATGGCATGACAGTAAGAGAATTATGCAGTGCTGC
CATAACCATGAGTGATAACACTGCGGCCAACTTACTTCTGACAACGATCGGA
GGACCGAAGGAGCTAACCGCTTTTTTGCACAACATGGGGGATCATGTAACTC
GCCTTGATCGTTGGGAACCGGAGCTGAATGAAGCCATACCAAACGACGAGCG
TGACACCACGATGCCTGTAGTAATGGTAACAACGTTGCGCAAACTATTAACT
GGCGAACTACTTACTCTAGCTTCCCGGCAACAATTAATAGACTGGATGGAGG
CGGATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGCAG
CACTGGGGCCAGATGGTAAGCCCTCCCGTATCGTAGTTATCTACACGACGGG
GAGTCAGGCAACTATGGATGAACGAAATAGACAGATCGCTGAGATAGGTGCC
TCACTGATTAAGCATTGGTAACTGTCAGACCAAGTTTACTCATATATACTTTA
GATTGATTTAAAACTTCATTTTTAATTTAAAAGGATCTAGGTGAAGATCCTTT
TTGATAATCTCATGACCAAAATCCCTTAACGTGAGTTTTCGTTCCACTGAGCG
TCAGACCCCGTAGAAAAGATCAAAGGATCTTCTTGAGATCCTTTTTTTCTGCG
CGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGA
GCGCAGATACCAAATACTGTCCTTCTAGTGTAGCCGTAGTTAGGCCACCACTT
CAAGAACTCTGTAGCACCGCCTACATACCTCGCTCTGCTAATCCTGTTACCAG
TGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTG
AGCTATGAGAAAGCGCCACGCTTCCCGAAGGGAGAAAGGCGGACAGGTATC
CGGTAAGCGGCAGGGTCGGAACAGGAGAGCGCACGAGGGAGCTTCCAGGGG
GAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCA
GCAACGCGGCCTTTTTACGGTTCCTGGCCTTTTGCTGCGGTTTTGCTCACATGT
TCTTTCCTGCGTTATCCCCTGATTCTGTGGATAACCGTATTACCGCCTTTGAGT
GAGCTGATACCGCTCGCCGCAGCCGAACGACCGAGCGCAGCGAGTCAGTGAG
CGAGGAAGCGGAAG

### Key:

Underlined font: CMV Enhancer/promoter
Bold font: Kozak sequence
Underlined and italic font: RPE65 gene

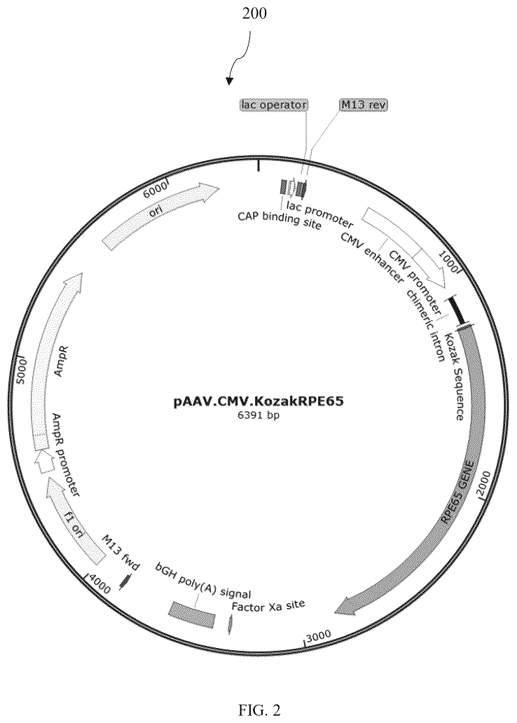

FIG. 2 is a schematic representation of pAAV.CMV.Kozak.RPE65, in accordance with an embodiment of the present invention.

In another embodiment of the present invention, a process for preparing an optimized transgene is provided. The process includes developing optimized RPE65 transgene with capsid modified AAV2 RPE65 vectors.

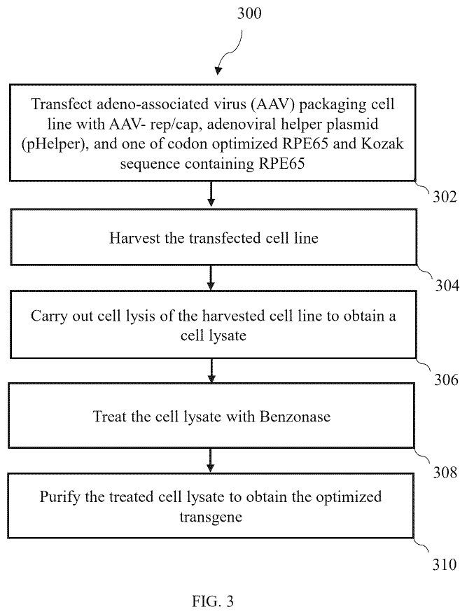

FIG. 3 represents a flowchart including steps of the process for preparing an optimized transgene, in accordance with an embodiment of the present invention. The process mainly involves transfection method. As used herein the term “transfection” refers to a method of introducing foreign DNA into the cells either by physical (electroporation) or chemical (cationic lipid or calcium phosphate reagents) methods.

The process for preparing an optimized transgene begins with transfecting adeno-associated virus (AAV) packaging cell line with AAV-rep/cap, adenoviral helper plasmid (pHelper), and one of codon optimized RPE65 and Kozak sequence containing RPE65 at step 302. The AAV-rep/cap comprises pAAV2K665Q/pAAV2K105Q.

In an embodiment, the transfected cell line is harvested after a duration of 72 hours at step 304. The transfected cell line is incubated for a duration of 72 hours. The incubation time after transfection vary depending on the goal of the experiment, nature of the plasmid used, and cell doubling time.

In an embodiment, cell lysis of the harvested cell line is carried out to obtain a cell lysate at step 306. The cell lysis helps in extracting genetic material from the harvested cell line. In one embodiment, the cell lysis is carried out by enzymatic, osmotic or mechanical disruption of the plasma membrane of a population of cells.

In an embodiment, the cell lysate is treated with Benzonase at step 308. In one embodiment, the cell lysate is treated with 25 units/ml of the Benzonase. The Benzonase is a genetically engineered endonuclease from Serratia marcescens. It degrades all forms of DNA and RNA (single stranded, double stranded, linear and circular) while having no proteolytic activity. It is effective over a wide range of conditions and possesses an exceptionally high specific activity. The Benzonase is utilized for separating DNA and RNA from proteins and other biologicals.

In such an embodiment, the treated cell lysate is purified to obtain the optimized transgene at step 310. The optimized transgene comprises one of a nucleotide sequence as set forth in SEQ ID No. 1 with pAAV.CMV.CodOpt.RPE65 and a nucleotide sequence as set forth in SEQ ID No. 2 with pAAV.CMV.Kozak.RPE65. The purifying the treated cell lysate is carried out by an iodixanol gradient ultracentrifugation followed by a column chromatography. The optimized transgene is concentrated and stored at −80° C.

In the present invention prepared optimized transgenes are characterized gene expression and immunocytochemistry etc. the characterization studies are as follows:

### 1. Quantitative PCR:

AAV genome titres are measured using quantitative real time-PCR. Samples are treated using DNase to remove non-encapsidated DNA. Encapsidated genome is targeted for amplification using polyadenylation (PolyA) primers. The PCR is performed on a CFX96 real-time PCR instrument (Bio-Rad, Hercules, CA, USA) using SYBR Green (Promega, Madison, WI, USA). Titres are generated from at least two biological replicates and the average value is calculated as vector genomes per milliliter (vgs/mL).

### 2. Relative Gene Expression In Vitro:

Huh-7 cells are seeded in a 24 well plate with a seeding density of 30,000 cells per well. Transduction is performed with AAV2K665Q CodOptRpe65/AAV2K665Q KozakRPE65/AAV2K105Q CodOptRPE65/AAV2K105Q KozakRPE65 at an MOI of 1×10⁵. Transfection is carried out with 2 μg of plasmid (pAAVRPE65WT/pAAVKozakRPE65/pAAVCodOptRPE65) using PEI as transfection agent (1:1). Total RNA from each experimental condition is extracted using TRIzol reagent (Thermo Fisher, Waltham, USA). cDNA is prepared from 1 μg of RNA using Verso cDNA synthesis kit (Thermo Fisher, Waltham, USA). qPCR is performed using RPE65 gene-specific primers for gene expression analysis. The qPCR data is analyzed using 2^{-ΔΔCt} method.

Table 1 Enlist sequences of the primers.

TABLE 1

| Primer Name | Forward Primers | Reverse Primers |
|---|---|---|
| CodOptRPE65 | AGCGAGCCTTTCTACCACCT | ATGGCTCTCACATAGGCGTC |
| KozakRPE65 | CATCCTGCTGGTGGTTACAA | ACAGGTGGTAAAATGGCTCA |
| Human β actin | AGTCCCTTGCCATCCTAAAAG | CAATGCTATCACCTCCCCTG |

### 3. Immunocytochemistry for RPE65:

Transduction is performed in Huh7 cells seeded in 8 well-chambered coverslips. At 48 hours post-transduction, the cells are fixed with 3% paraformaldehyde for 10 min. Afterwards, coverslips are rinsed with phosphate-buffered saline (PBS) and blocking is performed using 3% bovine serum albumin (BSA) and 0.2% Triton-X 100 in PBS for 1 hour. Coverslips are incubated with anti-RPE65 monoclonal antibody (1:250) (Abcam, UK) for 1 hour. After three washes, the coverslips are incubated with secondary antibody anti-rabbit CY3 (1:200) (Jackson ImmunoResearch, USA) for 40 min. Following three washes, the cells are counter-stained with 4′,6-diamidino-2-phenylindole (DAPI) (1:10,000) (Sigma-Aldrich, USA) and mounted with FluorSave™ (Sigma-Aldrich). The fluorescent signals are observed under 20× objective using LSM780NLO confocal microscope system, Carl Zeiss.

4. In-Vivo RPE65 Vector Administration in rd12 Mice:

Animals are anaesthetized with a ketamine/xylazine solution. Once unconscious, 1% tropicamide is applied to dilate the pupil and a small incision on the limbus is made using a 30G needle. Subretinal injection is performed using a hamilton syringe at a vector dose of 1×10⁹ vgs/eye in 6-8-week-old rd12 mice with 3-5 animals in each group (Mock, AAV2K665Q CodOptRPE65, AAV2K665Q KozakRPE65, AAV2K105Q CodOptRPE65 and AAV2K105Q KozakRPE65). Animals are followed-up till 10 weeks.

### 5. Electroretinography:

Scotopic ERG is performed using a Ganzfeld ERG system (Phoenix Research Labs. ERG is measured at 10-weeks post vector administration. Mice are dark adapted for overnight and anesthetized using ketamine/xylazine solution. A reference electrode is placed in the center of the scalp, and a ground electrode was set in the proximal portion of the tail skin. The pupils are dilated by eye drops containing a mixture of 0.5% tropicamide and 0.5% phenylephrine hydrochloride. The light stimulus is applied at varying light intensities ranging from −1.7 to 3.1 log cd sec/m².

### 6. Immunohistochemistry for RPE65 and GFAP Expression:

For immunostaining of retinal sections, mice are enucleated after 17 weeks of gene transfer. Eyes are cryo-sectioned and retinal sections are fixed in 4% paraformaldehyde for 15 mins, followed by incubation with blocking buffer for 2 hours. The retinal sections then are incubated with an anti-RPE65 antibody (1:250, Abcam, Cambridge, UK) and further stained by 1:200 goat anti-rabbit cy3 antibody. For nuclear staining, 4, 6-diamidino-2-phenylindole (DAPI) is used at 1:1000 dilution. The retinal sections are imaged by confocal microscopy. Further, the expression of glial fibrillary acidic protein (GFAP) is also studied by immunostaining using anti-GFAP (1:250, Cell signaling technologies, Danvers, USA) and counterstained with goat anti-rabbit cy3 antibody (1:200, Jackson ImmunoResearch, West Grove, USA).

### 7. Statistical Analysis:

Statistical analysis is performed using either one way ANOVA unpaired two-tailed Student's t test. A p value≤0.05 is considered statistically significant. All analysis is performed using GraphPad Prism 7.0 (GraphPad Software, La Jolla, CA, USA).

### Results:

### Enhanced Expression of CodOptRPE65 in Huh7 Cells:

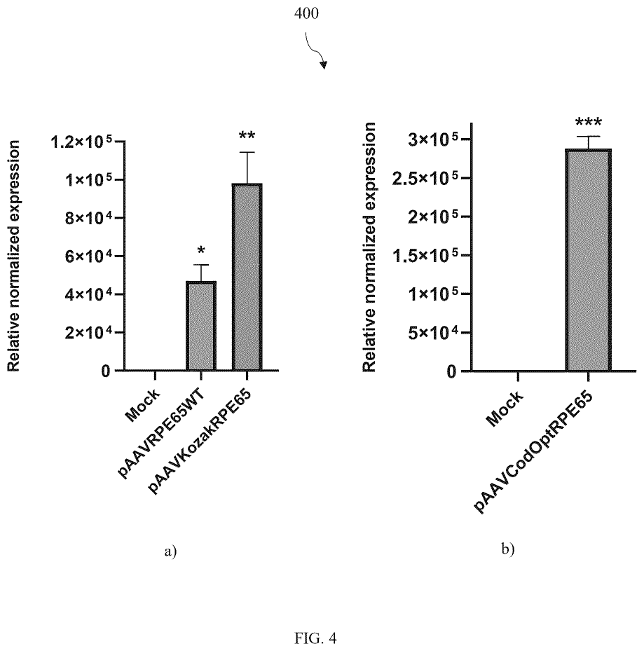

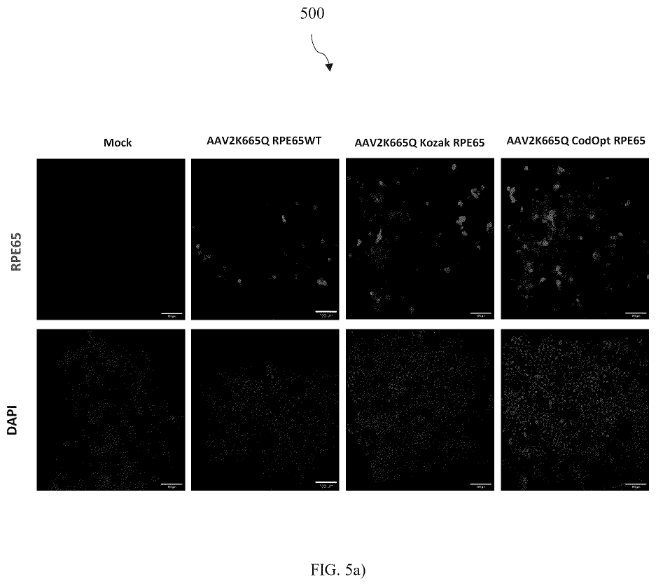

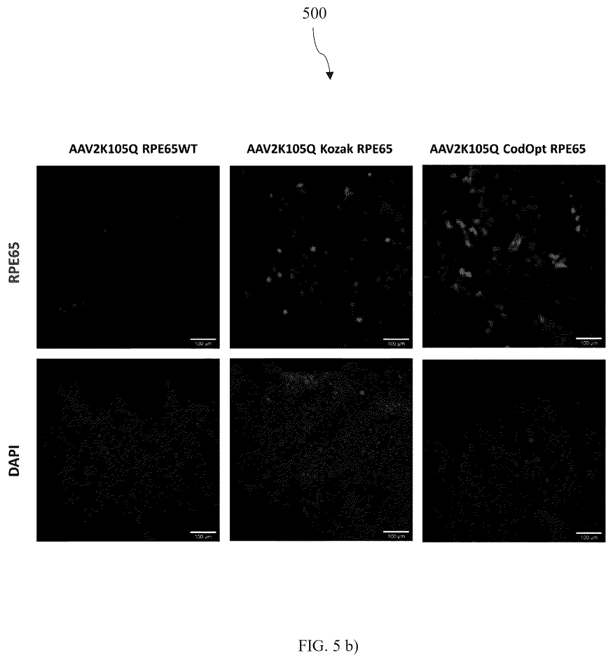

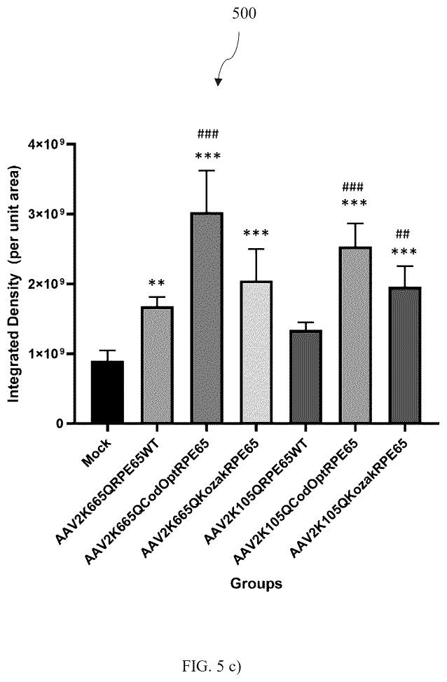

Relative normalized gene expression showed a significant increase in transcript levels in AAV-Rpe65 transfected cells compared to mock-treated cells. In comparison to pAAVRPE65WT, a 2.08-fold increase in pAAVKozakRPE65 transfected cells is observed. With codon optimized gene specific primer set, a high RPE65 expression in pAAVCodOptRPE65 transfected cells compared to the mock condition is observed (FIG. 4).

FIG. 4 is a graphical representation of relative RPE65 gene expression for pAAVKozakRPE65 and pAAVCodOptRPE65, in accordance with an embodiment of the present invention. The RPE65 gene expression is studied for two targets CodoptRPE65 and KozakRPE65 using specific primer sets. The comparison is presented with respect to mock-treated cells. Human β actin is taken as the reference gene for both targets. Data are mean±SD (n=3 replicates each condition, *p<0.05, **p<0.01, ***p<0.001, in comparison to mock-treated cells).

### Immunocytochemistry for RPE65 Expression in Huh7 Cells:

The subcellular localization of RPE65 is examined by immunocytochemistry using anti-RPE65 antibody. In transduced cells, a significant increase in AAV2K665Q KozakRPE65, AAV2K665Q CodoptRPE65, AAV2K105Q KozakRPE65 and AAV2K105Q CodOptRPE65 when compared to wild type RPE65 treated cells (FIG. 5a-5b) is observed. Upon quantification of the fluorescence intensity for RPE65 expression, the SUMOylation-site mutant vectors (AAV2 K105Q) containing KozakRPE65 showed ˜1.45-fold increase in RPE65 expression whereas, CodOptRPE65 showed ˜1.88-fold enhanced RPE65 expression. In case of Neddylation-site mutant vector (AAV2K665Q) the CodOptRPE65 transgene exhibited ˜1.8-fold high RPE65 and KozakRPPE65 showed ˜1.22-fold expression when compared to RPE65 WT vectors (FIG. 5c). The immunocytochemistry data suggested that the optimized (Kozak or codon optimized) transgenes exhibited higher RPE65 expression when compared to the wild type RPE65. On the basis of this finding, the optimized RPE65 constructs with mutant AAV2 serotypes are tested in vivo for their therapeutic efficacy.

FIG. 5 represents immunostaining of RPE65 a-b) RPE65 expression post-transduction and c) quantification of integrated density per unit area, post-transduction, in accordance with an embodiment of the present invention. In (a-b) Scale bar is 100 μm. Cells are stained with an anti-RPE65 primary antibody and anti-rabbit Cy3 secondary antibody. Nuclei are counterstained using DAPI. In (c) * represents comparison with the mock sample, # represents comparison with respective (AAV2K105Q/AAV2K665Q) RPE65 WT vectors (*p<0.05, **p<0.01, ***p<0.001).

Improved Phenotypic Response in RPE65 Vector Administered rd12 Mice:

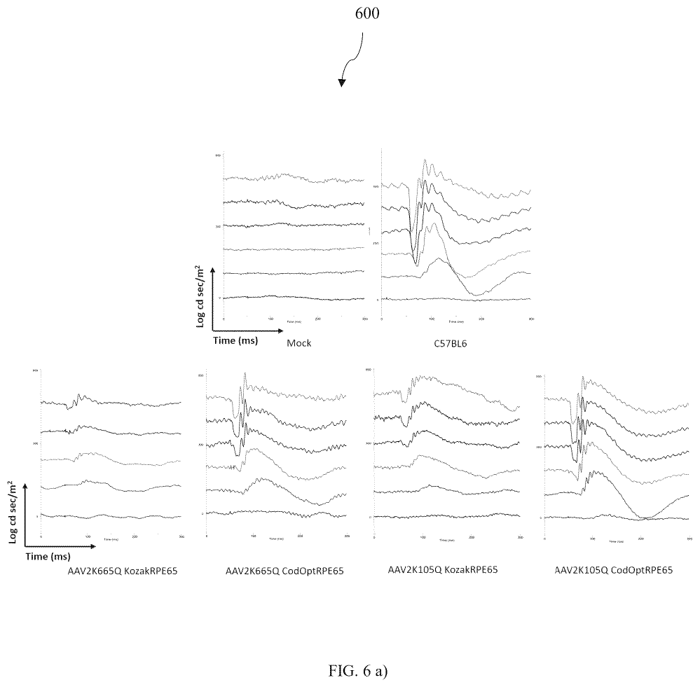

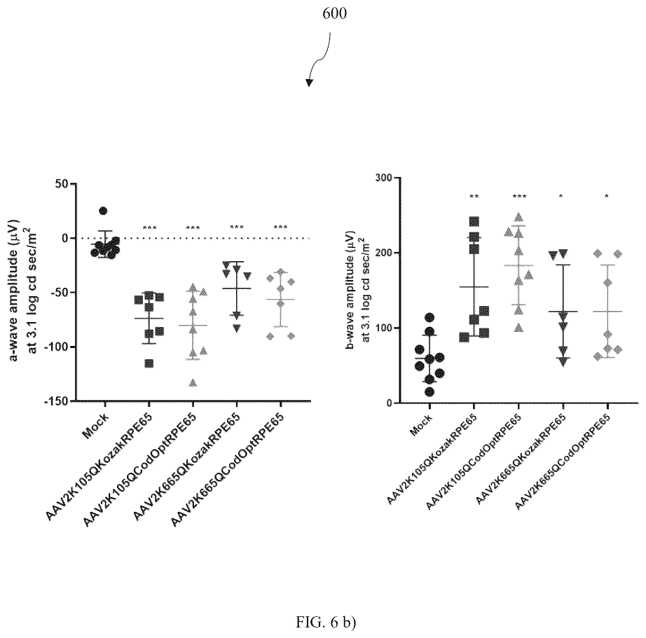

The phenotypic response is measured by scotopic electroretinography (ERG). Data is collected 10 weeks post vector administration using Ganzfeld ERG. The representative ERG waveforms from the treated mice (n=6-9 eyes per group) are shown in FIG. 6a. It is noted that a significant visual correction in eyes that received CodOptRPE65 and KozakRPE65 vectors, with a significant increase in ‘A-wave’ and ‘B-wave’ amplitude in comparison to the un-injected mock animals (FIG. 6b).

FIG. 6 represents ERG for test groups after 10 weeks follow-up a) representative ERG waterfall from each group and b) graphical representation of ‘A-wave’ and ‘B-wave’ amplitude, in accordance with an embodiment of the present invention. The ERG is recorded at varying light intensities ranging between −1.7 to 3.1 log cd sec/m² for varying time duration. Data are mean±SD. ANOVA based Dunett's test is used for statistical comparison between the groups. ns: p>0.05, *p<0.05, **p<0.01. A representative ERG from a normal [C57BL6] mice is shown for comparison.

### Immunohistochemical Analysis of RPE65 and GFAP Expression in Treated Eyes:

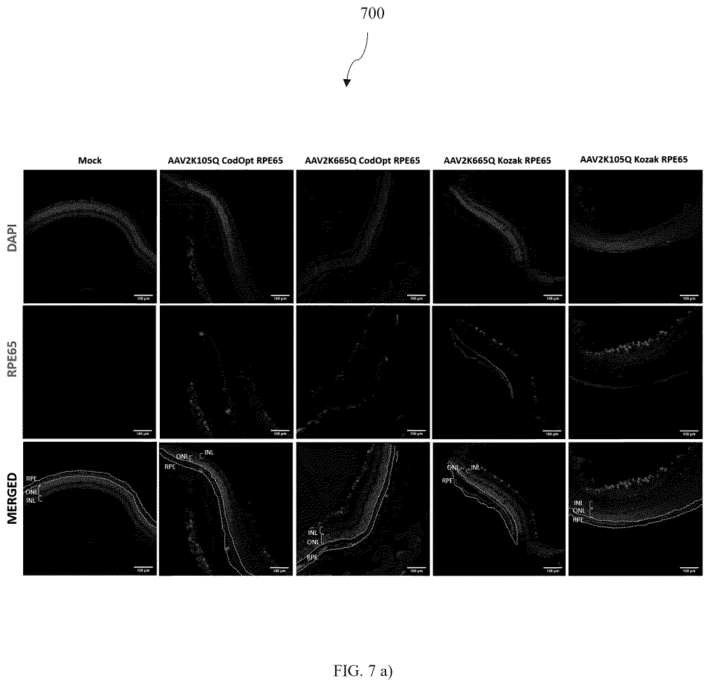

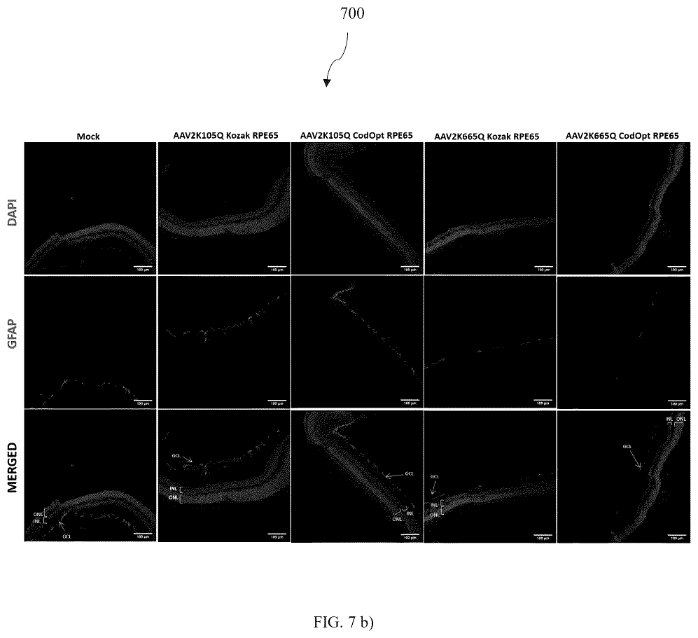

Rpe65 expression is confirmed by immunostaining of eye sections, 17-weeks post vector administration in the treated group as compared to the mock group (FIG. 7a). GFAP staining in treated animals showed similar expression to mock group signifying the absence of vector-induced inflammation in treated animals (FIG. 7b).

FIG. 7 represents immunostaining of mice eye sections a) RPE65 and b) GFAP staining, in accordance with an embodiment of the present invention. About 10 μM cryosections of eyes from all groups are stained with anti-RPE65 (1:250, Abcam, Cambridge, UK) and counterstained with goat anti-rabbit Cy3 (1:200, Abcam) antibody or with anti-GFAP (1:200, Cell signaling technologies, Danvers, USA) and counterstained with goat anti-rabbit cy3 antibody (1:200, Jackson ImmunoResearch, West Grove, USA). In the images Scale bar is 100 μm, “GCL” represents ganglion cell layer, “INL” represents inner nuclear layer, “ONL” represents outer nuclear layer, and “RPE” represents retinal pigment epithelium (marked with dotted line).

LCA2 is a congenital dystrophy that results in severe vision loss at an early age. In the present invention, gene therapy has been used to restore vision in RPE65 deficient mice model using viral vectors. To overcome the limitations of the current therapy, generation of a modified vector can provide enhanced therapeutic effects. Codon optimization is known to result in strong upregulation of protein and RNA levels, indicating the importance of codon usage in determining a gene's expression. On the other hand, the kozak sequence guides the ribosome in which AUG is used as initiator AUG for the translation. Hence, addition of the kozak sequence is expected to increase the translation efficiency of the upstream sequence.

In the present invention, increase in the transcript levels of CodOptRPE65 is observed when compared to KozakRPE65. The immunocytochemistry also showed enhanced RPE65 expression in CodOptRPE65 when compared to KozakRPE65 suggesting a high level of RPE65 protein expression.

The in vivo study revealed that animals treated with CodOptRPE65 and KozakRPE65 transgenes under modified capsids, show significant improvement in vision rescue, with an improved impact of pAAV.CMV.CodOpt.RPE65 vectors, when compared to mock treated animals. This observation is validated after RPE65 immunostaining of eyes. There was no effect of the vector in the treated groups on GFAP expression, signifying that there is no vector induced immune response in the eye. Hence, optimized forms of RPE65 transgene could be potentially used for enhanced phenotypic rescue in LCA2 patients.

The present invention provides the system including the one or more optimized transgene for ocular gene therapy. The one or more optimized transgene including codon optimized and Kozak driven RPE65 vectors improve the efficiency as demonstrated in suitable models of the ocular gene therapy for LCA2. The one or more optimized transgenes provides enhanced therapeutic response when compared to the gene transfer of wild type RPE65 gene. The present invention also provides the process for preparing optimized transgene. The process provided by the present invention is simple and efficient.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A system for an ocular gene therapy, comprising:
one or more optimized transgenes configured to improve efficiency of the ocular gene therapy in Leber congenital amaurosis 2 (LCA2) condition,
wherein the one or more optimized transgenes are selected from a group consisting of a nucleotide sequence as set forth in SEQ ID No. 1 with pAAV.CMV.CodOpt.RPE65 and a nucleotide sequence as set forth in SEQ ID No. 2 with pAAV.CMV.Kozak.RPE65.

2. A process for preparing an optimized transgene, comprising:
transfecting adeno-associated virus (AAV) packaging cell line with AAV-rep/cap, adenoviral helper plasmid (pHelper), and one of codon optimized RPE65 and Kozak sequence containing RPE65;
harvesting the transfected cell line after a duration of 72 hours;
carrying out cell lysis of the harvested cell line to obtain a cell lysate;
treating the cell lysate with Benzonase; and
purifying the treated cell lysate to obtain the optimized transgene,
wherein the optimized transgene comprises one of a nucleotide sequence as set forth with in SEQ ID No. 1 pAAV.CMV.CodOpt.RPE65 and a nucleotide sequence as set forth in SEQ ID No. 2 with pAAV.CMV.Kozak.RPE65.

3. The process as claimed in claim 2, wherein the AAV-rep/cap comprises pAAV2K665Q/pAAV2K105Q.

4. The process as claimed in claim 2, wherein the purifying the treated cell lysate is carried out by an iodixanol gradient ultracentrifugation followed by a column chromatography.

5. The process as claimed in claim 2, wherein the optimized transgene is concentrated and stored at −80° C.
